(19) United States
(12) Patent Application Publication
(10) Pub. No.: US 2010/0166804 A1
(43) Pub. Date: Jul. 1, 2010

(54) METHODS

(76) Inventor: Dennis Penn, Raleigh, NC (US)

Correspondence Address: HOXIE & ASSOCIATES LLC, 75 MAIN STREET , SUITE 301, MILLBURN, NJ, 07041, US

(21) Appl. No.: 12/600,510

(22) PCT Filed: May 23, 2008
(86) PCT No.: PCT/US08/06635

§ 371 Date: Nov. 17, 2009

Related U.S. Application Data
(60) Provisional application No. 60/931,556, filed on May 23, 2007.

(51) Int. Cl. A61K 39/35; A61K 31/4535

(52) U.S. Cl. 424/275.1; 514/324

(57) ABSTRACT

The present invention relates to methods of prophylactically treating, reducing, delaying or controlling severe allergic reaction, e.g., to food and/or hymenoptera allergen, e.g. peanut allergen or bee venom allergen, in patients at risk of systemic anaphylaxis, e.g., patients receiving allergen desensitization therapy, through the use of a mast cell stabilizer, e.g., ketotifen and/or its metabolites or derivatives in free or pharmaceutically acceptable acid addition salts thereof. The invention further provides methods to desensitize a patient to one or more allergens comprising administering (i) one or more allergen to induce tolerance and (ii) one or more mast cell stabilizers in free or pharmaceutically acceptable salt thereof.

This application claims priority from U.S. Provisional Application No. 60/931,556, filed May 23, 2007, the contents of which are hereby incorporated by reference.

### FIELD OF THE INVENTION

The present invention relates to methods of prophylactically treating, reducing, delaying or controlling severe allergic reactions, especially systemic anaphylaxis, including methods of providing allergen desensitization therapy with reduced risk of anaphylactic reaction.

### BACKGROUND

Over the past decade, concerns for the substantial increase in the prevalence of allergy have attracted much attention throughout the world. The various types of allergens that commonly cause allergic reactions include eggs, milk, soy, fish, shellfish, various types of fruits, seeds and nuts, penicillin, latex and insect venom. Among this group of allergens, peanuts, tree nuts and bee venom are most often associated with the severe allergic reaction known as anaphylactic shock. Allergy to peanut and/or tree nut is reported to affect 1.1% of the US population while venom of insect stings reportedly results in severe allergic reaction in 3% of the US population. These allergies affect individuals at all ages and while allergies to some of these food products such as milk may be outgrown, allergies to other food products such as peanuts, nuts and seafood are usually permanent. Among those reported for food allergens, approximately one third of the estimated 30,000 cases of food allergy requiring emergency room treatment for the life-threatening anaphylactic shock are attributed to peanut and tree nut products. Though deaths resulting from anaphylactic shocks are low, 90% of the deaths that did occur are due to peanut allergy. Therefore, there exists a growing need for prophylactic treatment or control of systemic anaphylaxis due to allergy. In particular, there exists a growing need for prophylactic treatment or control of systemic anaphylaxis due to food and hymenoptera allergy, particularly allergy to peanut and bee venom.

The term “anaphylaxis” is commonly used to refer to a Type I allergic or hypersensitive reaction of the immune system. This reaction occurs when antigens or allergens bind to immunoglobulin E (IgE) antibodies that in turn bind to the receptors on mast cells and trigger the mast cell to release histamine and leukotrienes, causing blood vessels to dilate and airways to narrow. Most people with type I allergy manifests a milder form of anaphylaxis called local anaphylaxis, which is a localized reaction to antigen or allergen that results in urticaria (hives), pruritis (itchiness), mild asthma, hay fever, and/or dermatitis. However, a relatively small number of individuals with type I allergy manifests a much more severe form of anaphylaxis, called systemic anaphylaxis, where the antigens or allergens trigger an explosive mast cell release of histamine and leukotrienes throughout the body, causing swelling of the lips, tongue, upper airway as well as leakage of the capillary, leading to life-threatening collapse of the respiratory and circulatory system. Though medical attention has been directed towards the more commonly occurred type I allergy such as urticaria, asthma (particularly allergic asthma) and pruritis, prophylactic treatment and/or control of the more severe type I allergy, systemic anaphylaxis, through the use of a mast cell inhibiting agent is still unavailable. Although epinephrine is the principal emergency treatment for anaphylaxis, there is also a growing need for prophylactic treatment to reduce the risk of systemic anaphylaxis, especially for patients who have developed resistance to epinephrine, e.g., as a result of continuous use of long-acting β-adrenergic antagonists or β-agonists.

Various prophylactic treatment options are available or being developed. See Pons et al., “Towards immunotherapy for peanut allergy,” Curr. Opin. Allergy Clin. Immunol., 5:558-562 (2005). Injection immunotherapy has been used to treat food allergy, however, it is not recommended because of allergic side effects of the therapy. Unacceptably high rates of adverse systemic reactions are associated with the therapy and maintenance protocols.

### SUMMARY OF THE INVENTION

The present invention addresses an unmet, yet much-needed prophylactic treatment or control of severe reaction to allergens, particularly food or hymenoptera allergens, e.g., peanut allergens or bee venom, in patients at risk of life-threatening, systemic anaphylaxis. The present invention also provides a method of ameliorating side effects of oral, inhaled or injection immunotherapy. Therefore, in one embodiment, the invention provides Method I for prophylactically treating, delaying, controlling and/or reducing severe reaction to allergens in a patient at risk of systemic anaphylaxis if exposed to such allergens, comprising administering to said patient an effective amount of ketotifen in free or pharmaceutically acceptable acid addition salt form.

The invention further provides for the following methods:
1.1 Method I, wherein said patient has a history of at least one systemic anaphylactic reaction.
1.2 Method I or 1.1, wherein said patient has a co-existing condition of asthma.
1.3 Method I, 1.1 or 1.2, wherein said patient has an elevated IgE level.
1.4 Method I, 1.1, 1.2 or 1.3, wherein said patient has an elevated basal tryptase level.
1.5 Method I, 1.1, 1.2, 1.3 or 1.4, wherein said patient has a B16-Arg/Arg genotype.
1.6 Method I or any of methods 1.1-1.5, wherein said level of IgE is greater than 30 kU/L.
1.7 Method I or any of methods 1.1-1.6, wherein said basal tryptase level is greater than 13 m/L.
1.8 Method I or any of methods 1.1-1.7, wherein said patient has a skin-prick test (SPT) wheal diameter of greater than 8 mm.
1.9 Method I or any of methods 1.1-1.8, wherein said patient is unwilling to undergo or unable to respond to desensitization treatment.
1.10 Method I or any of methods 1.1-1.9, wherein said acid addition salts are selected from a group consisting of hydrochloride, hydrobromide, hydrogen sulfate, phosphate, maleate, nitrate, besylate and fumarate salts.
1.11 Method I or any of methods 1.1-1.10, wherein said acid addition salt is the fumarate salt.
1.12 Method I or any of methods 1.1-1.11, further comprises administering said ketotifen in free or pharmaceutically acceptable acid addition salt thereof in conjunction with at least one therapeutic agents selected from a group consisting of histamine-1 receptor antagonists, histamine-2 receptor antagonists, corticosteroids, PDE IV inhibitors, beta-adrenergic agonists, anticholinergic agents, epinephrine and IgE antagonists (including variant anti-IgE antibodies (non-human mammalian, chimeric, polyclonal and/or monoclonal antibodies (Mabs) and fragments thereof (e.g., proteolytic digestion or fusion protein products) and nucleotides or polypeptides encoding or containing IgE sequences or nucleotides or polypeptides that induce anti-IgE antibody production (e.g., anti-IgE immunogenic polypeptide as disclosed in U.S. Pat. No. 6,913,749, U.S. Pat. App. No. 2004/0076625A1 and 2003/0031663A1), especially IgE antagonist and polypeptides capable of differential binding to Fc.epsilon.RI and Fc.epsilon.RII.); antagonist of cytokines, such as, for example, monoclonal antibody to TNF-alpha, soluble TNF-alpha receptor-Fc fusion protein, and/or IL-1 receptor; kinase inhibitors, e.g., inhibitors of spleen tyrosine kinase (syk), and inhibitors of kinase-activated pathways, e.g. inhibitors of STAT proteins.
1.13 Any of the preceding methods, wherein said patient has a history of at least one severe bronchospasm in the absence of prior indication of asthma.
1.14 Any of the preceding methods, wherein said patient is under the age of 15 and has a personal history of food allergy and a family history of anaphylaxis.
1.15 Any of the preceding methods wherein said patient has diminished or inadequate response to β₂-adrenergic agonists.
1.16 Any of the preceding methods wherein said patient is resistant to epinephrine treatment.
1.17 Any of the preceding methods wherein the patient is a regular user of long acting beta-agonists (LABAs)
1.18 Any of the preceding methods wherein said patient is a regular user of β₂-adrenergic antagonist.
1.19 Any of the preceding methods wherein said patient has an IgE level of greater than 30 kU/L.
1.20 Any of the preceding methods wherein said patient has a basal tryptase level of greater than 13 μg/L.
1.21 Any of the preceding methods wherein the allergen is selected from allergens in milk, eggs, peanuts, tree nuts, fish, shellfish, soy and wheat, or from allergens in hymenoptera venom.
1.22 Any of the preceding methods wherein the ketotifen is administered in an oral dose of 0.01-0.1 mg/kg twice daily.
1.23 Any of the preceding methods wherein the patient receives an oral dose of 0.5-4 mg ketotifen twice daily.
1.24 Any of the preceding methods wherein the patient receives an oral dose of 0.5, 1 or 2 mg ketotifen twice daily.
1.25 Any of the preceding methods wherein the patient receives an oral dose of 1-6 mg of ketotifen once a day.
1.26 Any of the preceding methods wherein the patient receiving an oral dose of ketotifen requires smaller doses of Anti-IgE antibody.
1.27 Any of the proceeding methods wherein the patients receiving an oral dose of ketotifen require a smaller dose of antagonist of cytokines, such as, for example, monoclonal antibody to TNF-alpha, soluble TNF-alpha receptor-Fc fusion protein, and/or IL-1 receptor, to provide effective treatment for chronic inflammatory diseases.
1.28 Any of the proceeding methods wherein the patients receiving an oral dose of ketotifen require a smaller dose of corticosteroid.
1.29 Any of the preceding methods wherein the allergen is from peanuts.
1.30 Any of methods 1-1.28 wherein the allergen is from hymenoptera venom.
1.31 Method 1.30 wherein the allergen is from bee, wasp, hornet, fire ant or yellow jacket venom.
1.32 Method 1.31 wherein the allergen is from bee venom.
1.33 Any of the preceding methods wherein the patient is undergoing allergen desensitization therapy.
1.34 Any of the preceding methods wherein the patient is undergoing oral allergen desensitization therapy.
1.35 Any of the preceding methods wherein the patient is undergoing sublingual allergen desensitization therapy.
1.36 Any of the preceding methods wherein the patient is undergoing injection allergen desensitization therapy.
1.37 Any of the preceding methods wherein the patient is undergoing allergen desensitization therapy wherein the allergen is administered via inhalation (e.g., airborne allergens).
1.38 Any of the preceding methods wherein the patient has a co-morbid disease.
1.39 Method 1.38 wherein said co-morbid disease is allergic rhinitis or dermatitis.
1.40 Any of methods 1.1-1.20, 1.22-1.28, and 1.34-1.39, wherein the allergen is dust mites.
1.41 Any of the preceding methods wherein the allergen is allergen from bee venom.
1.42 Any of the preceding methods wherein the allergen is food allergen.
1.43 Any of the preceding methods wherein the allergen is peanut allergen.
1.44 Any of the preceding methods wherein the allergen is administered orally, sublingually, via inhalation or injection.
1.45 Any of the preceding methods wherein the patient is an infant, a child or an adolescent.
1.46 Any of methods 1.33-1.45, wherein the allergen is administered in increasing doses over a shorter period of time than when administered in the absence of the administration of a mast cell stabilizer.

In a preferred embodiment, the Invention provides Method I, e.g., any of methods 1.1-1.46, for the prophylactic treatment, delay, control or reduction of severe reaction to peanut allergen in a patient at risk of systemic anaphylaxis comprising administering to said patient an effective amount of ketotifen fumarate.

In another preferred embodiment, the Invention provides Method I, e.g., any of methods 1.1-1.46, for the prophylactic treatment, delay, control or reduction of severe reaction to bee venom in a patient at risk of systemic anaphylaxis comprising administering to said patient an effective amount of ketotifen fumarate.

In yet another preferred embodiment, the Invention provides Method I, e.g., of any of methods 1.1-1.46, wherein said patient has diminished or inadequate response to β₂-adrenergic agonists, for example, a patient who
a) is homozygous for a codon encoding arginine at position 16 on the beta-2 receptor;
b) is a regular user of beta-agonists, e.g., long-acting beta-agonists; or
c) is a regular user of beta-antagonists.

In still another particular embodiment, the invention provides the prophylactic treatment, delay, control or reduction of severe reaction to peanut allergen in a patient at risk of systemic anaphylaxis comprising administering to said patient an effective amount of ketotifen in free or pharmaceutically acceptable acid additional salt form, optionally in conjunction with at least one therapeutic agents selected from a group consisting of histamine-1 receptor antagonists, histamine-2 receptor antagonists, corticosteroids, PDE IV inhibitors, beta-adrenergic agonists, anticholinergic agents, epinephrine and IgE antagonists (including variant anti-IgE antibodies (non-human mammalian, chimeric, polyclonal and/or monoclonal antibodies (Mabs) and fragments thereof (e.g., proteolytic digestion or fusion protein products) and nucleotides or polypeptides encoding or containing IgE sequences or nucleotides or polypeptides that induce anti-IgE antibody production (e.g., anti-IgE immunogenic polypeptide as disclosed in U.S. Pat. No. 6,913,749, U.S. Pat. App. No. 2004/0076625A1 and 2003/0031663A1), especially IgE antagonist and polypeptides capable of differential binding to Fc.epsilon.RI and Fc.epsilon.RII.); antagonist of cytokines, such as, for example, monoclonal antibody to TNF-alpha, soluble TNF-alpha receptor-Fc fusion protein, and/or IL-1 receptor; kinase inhibitors, e.g., inhibitors of spleen tyrosine kinase (syk), and inhibitors of kinase-activated pathways, e.g. inhibitors of STAT proteins.

In a another embodiment, the invention provides Method II, a method for desensitizing a patient to an allergen comprising, administering to the patient an allergen and an effective amount of one or more mast cell stabilizers in free or pharmaceutically acceptable acid addition salt form to inhibit symptoms of mediator release (e.g., decrease in blood pressure, angioedema, urticaria, etc.). The mast cell stabilizers useful for the present invention may be cromolyn, cromoglycate, nedocromil, bepotastine, ebastine, epinastine, azelastine, lodoxamide tromethamine, pemirolast, olopatadine, ketotifen, norketotifen, 10-OH-norketotifen, 9-OH-norketotifen, 9,10-di-OH-norketotifen, or other ketotifen metabolites or derivatives thereof, in free or pharmaceutically acceptable acid addition salt form. Other mast cell stabilizers useful for the present invention may also include:

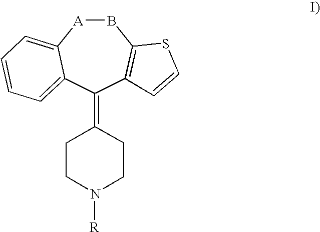

wherein:
a) -A-B— is a moiety having the formula:
   i) —CO—CH₂—
   ii) —CH₂—CO—
   iii) —CH₂—CH₂—
   iv) —CHOH—CH₂—
   v) —CHOH—CHOH—
   vi) —CH₂—CHOH—; or
   vii) —CO—CO—; and
b) R is a hydroxyalkyl or a carboxyalkyloxyalkyl moiety;

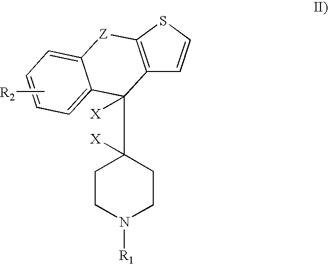

wherein:
a) —Z— is —CH₂—CH₂— or —CH═CH—;
b) R₁ is a H or C₁₋₄alkyl; and
c) R₂ is H or halogen.

or

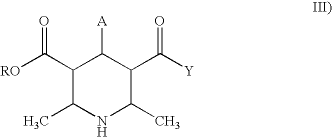

wherein:
a) A is

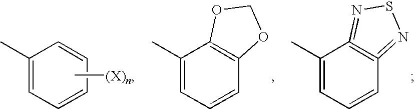

b) n is 1 or 2;
   when n is 1, X is H, halogen, CF₃, alkyne, —S(O)_{n′}R, OR or NO₂;
   when n is 2, X is H or halogen;
c) n′ is 0, 1 or 2;
d) R is C₁₋₅(un)branched alkyl;
e) Y is Z(C(R′)(R′))_{n′}Z′ or

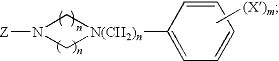

wherein:
i) n″ is 2-4;
ii) m is 0, 1, 2, 3;
iii) R′ is H or R;
iv) X′ is H, halogen, CF₃, alkyne, —S(O)_{n″}R, OR or NO₂;
v) Z is NH or O;
vi) Z′ is N(R′)(R″) or

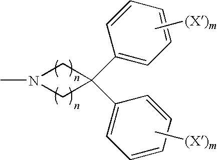

or R″ is

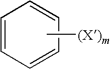

in free or pharmaceutically acceptable acid addition salt form.

More preferably, the mast cell stabilizer of the invention is ketotifen, most preferably ketotifen fumarate. The invention therefore provides a method for desensitizing a patient to an allergen comprising, administering to the patient an allergen and an effective amount of ketotifen in free or pharmaceutically acceptable acid addition salt form to inhibit symptoms of mediator release (e.g., decrease in blood pressure, angioedema, urticaria, etc.).

The invention also provides Method II in combination with any of Methods 1-1.8 and 1.10-1.46 (Method II-A). Therefore, in one embodiment, Method II-A is a method to desensitize a patient to an allergen comprising administering an allergen and one or more mast cell stabilizer, e.g., ketotifen in free or pharmaceutically acceptable salt form, to a patient in need thereof wherein said patient has a co-existing condition of asthma or said patient is a regular use of beta-antagonists or beta-agonists, e.g., long-acting beta-agonists. In another embodiment, Method II-A may also include a method to desensitize a patient to an allergen comprising administering an allergen and one or more mast cell stabilizer, e.g., ketotifen in free or pharmaceutically acceptable salt form, optionally in conjunction with at least one therapeutic agents selected from a group consisting of histamine-1 receptor antagonists, histamine-2 receptor antagonists, corticosteroids, PDE IV inhibitors, beta-adrenergic agonists, anticholinergic agents, epinephrine and IgE antagonists (including variant anti-IgE antibodies (non-human mammalian, chimeric, polyclonal and/or monoclonal antibodies (Mabs) and fragments thereof (e.g., proteolytic digestion or fusion protein products) and nucleotides or polypeptides encoding or containing IgE sequences or nucleotides or polypeptides that induce anti-IgE antibody production (e.g., anti-IgE immunogenic polypeptide as disclosed in U.S. Pat. No. 6,913,749, U.S. Pat. App. No. 2004/0076625A1 and 2003/0031663A1), especially IgE antagonist and polypeptides capable of differential binding to Fc.epsilon.RI and Fc.epsilon.RII.); antagonist of cytokines, such as, for example, monoclonal antibody to TNF-alpha, soluble TNF-alpha receptor-Fc fusion protein, and/or IL-1 receptor; kinase. The allergen may be administered with one or more mast cell stabilizers over a period of time, whereby the dose of the allergen administered to said patient is increased over the period of time. For example, the mast cell stabilizer may be administered prior to the administration of each dose of allergen. Preferably, the mast cell stabilizer is administered up to 2 weeks prior to the administration of each dose of allergen. More preferably, the mast cell stabilizer is administered up to 24 hours prior to the administration of each dose of allergen. Thus, the administration of the allergen and ketotifen may allow for larger doses of the allergen to be administered than administration of the allergen alone, i.e., the patient is able to tolerate greater amounts of allergen. Thus the method for desensitization is available to patients who are unwilling to undergo or unable to respond to normal desensitization treatment.

The method for desensitization may also shorten or expedite the desensitization process due to the patience's higher tolerance for the allergen as a result of the mast cell stabilizers. Therefore, in another embodiment, the invention provides a method (Method III) for expediting allergy desensitization comprising Method II or in combination with any of Methods 1-1.8 and 1.10-1.46, wherein the amount of allergen administered to induce tolerance. The amount of allergen administered for expedited desensitization (or RUSH immunotherapy) is higher than would be administered to the patient in the absence of co-administration of a mast cell stabilizer. In another embodiment, the frequency of administration of the allergen is higher than the frequency where allergen is not administered in conjunction with a mast cell stabilizer such as ketotifen. For example, a patient undergoing desensitization may begin with RUSH immunotherapy (i.e., expedited desensitization) wherein said patient receives on day one a series of small, escalating doses of allergen in conjunction with (e.g., during, before or after the administration of) the mast cell stabilizer to inhibit or prevent mediator release. Said patient then receives increasing doses of allergen at a slower frequency so as to build-up to a maintenance dose over a period of time. The maintenance dosage and the period of time during which the maintenance doses are administered may vary depending on each patient and said patient's sensitivity to the allergen and may last for years or even indefinitely. By undergoing RUSH immunotherapy with the use of the mast cell stabilizer, however, said patient may be able tolerate larger and/or more frequent doses of allergen with fewer adverse events, thereby, enhancing patient compliance with desensitization protocol, improving efficacy, and reducing the number of doctor's office visits.

In another embodiment, the mast cell stabilizer is administered only in response to an allergic reaction arising from administration of the allergen (Method IV). Therefore, the mast cell stabilizer is administered after the administration of an allergen, e.g., after manifestation of an allergic reaction. The allergen may be administered by any suitable route, preferably by the subcutaneous, oral, inhalation or sublingual route.

In a further embodiment, the invention provides any of Methods II, II-A, III or IV, wherein said method desensitizes said patient from more than one allergen, e.g., desensitizes said patient from a combination of peanut and hymenoptera and other allergens.

In a still further embodiment, the invention provides any of Methods II, II-A, III or IV, wherein said patient suffers from a co-morbid disease, e.g., allergic rhinitis, dermatitis, or asthma.

The invention also provides a pharmaceutical composition comprising ketotifen in free or pharmaceutically acceptable acid addition salt form for use in Method I e.g., in any of methods 1.1-1.46 or in Method II. The pharmaceutical compositions are preferably in admixture with a pharmaceutically acceptable diluent or carrier. In another embodiment, the invention provides pharmaceutical compositions comprising a mast cell stabilizer in free or pharmaceutically acceptable acid addition salt form in admixture with a pharmaceutically acceptable diluent or carrier for use in any of Methods II, II-A, III or IV.

In a further embodiment, the invention provides use of ketotifen in free or pharmaceutically acceptable acid addition salt form, e.g., of ketotifen fumarate, in the manufacture of a medicament for the prophylactic treatment, delay, control and/or reduction of severe reaction to food allergen or symptoms of mediator release during allergen desensitization in a patient at risk of systemic anaphylaxis, e.g., in the manufacture of a medicament for use in Method I as described above, e.g., any of methods 1.1-1.46 or Method II. In another embodiment, the invention provides use of a mast cell stabilizer in free or pharmaceutically acceptable acid addition salt thereof in the manufacture of a medicament for the desensitization of a patient to one or more allergen, e.g., food, hymenoptera, tree or plant allergen, comprising administering (i) one or more allergen to induce tolerance and (ii) one or more mast cell stabilizers in free or pharmaceutically acceptable acid addition salt form to inhibit or prevent any symptom of mediator release, e.g., in any of Methods II, II-A, III, IV, V or VI. In a particular embodiment, the mast cell stabilizer is ketotifen fumarate. In another embodiment, invention provides any of the foregoing methods wherein the allergen comprises allergens from various sources such as peanut are administered concomitantly, e.g., as an admixture; separately but simultaneously or concurrently; or sequentially.

In still another embodiment, the invention provides a method of allergen desensitization of a patient to one or more allergen, e.g., food (e.g., peanut, tree nuts), hymenoptera venom (e.g., bee venom), drugs (e.g., β-lactam antibiotics, insulin (e.g., Humulin, Novolin), streptokinase, allergen extracts), and/or airborne allergen such as plant allergen (e.g., grass pollen, weed, ragweed, tree pollen, mold), dust, and/or animal or pet dander, comprising administering (i) one or more allergen to induce tolerance and (ii) one or more antihistamine in free or pharmaceutically acceptable acid addition salt thereof to inhibit or prevent any symptom of mediator release. The desensitization method of the invention also includes desensitization against allergen such as those found in dust mites. In yet another embodiment, the invention provides a method of allergen desensitization of a patient to one or more allergen, e.g., e.g., food (e.g., peanut, tree nuts), hymenoptera venom (e.g., bee venom), drugs (e.g., β-lactam antibiotics, insulin (e.g., Humulin, Novolin), streptokinase, allergen extracts), and/or airborne allergen such as plant allergen (e.g., grass pollen, weed, ragweed, tree pollen, mold), dust, dust mites, and/or animal or pet dander, comprising administering (i) one or more allergen to induce tolerance and (ii) one or more compound in free or pharmaceutically acceptable acid addition salt thereof, which compound has dual antihistamine and mast cell stabilizing activities to inhibit or prevent any symptom of mediator release. In a particular embodiment, such antihistamine is ketotifen. In yet another embodiment, the invention provides use of cromolyn, in conjunction with one or more leukotriene (LT) modulators, e.g., Zileuton (Zyflo®) or Montelukast (Singulair®), e.g., in any of Methods II, II-A, III, and IV.

In still another aspect, the invention provides a method of allergen desensitization comprising administering to a patient in need thereof (i) increasing doses of allergen to induce tolerance and (ii) an amount of at least one antihistamine to inhibit symptoms of mediator release (Method V). The antihistamine of Method V includes but not limited to ketotifen. Said antihistamine may be in free or pharmaceutically acceptable salt form, e.g., ketotifen fumarate.

In another aspect of the invention, there is provided a method of allergen desensitization comprising administering to a patient in need thereof (i) increasing doses of allergen to induce tolerance and (ii) an amount of a compound having dual antihistamine and mast cell stabilizing activities to inhibit symptoms of mediator release (Method VI). The compound of Method VI having a dual antihistamine and mast cell stabilizing activities include but not limited to ketotifen. Said compound may be in free or pharmaceutically acceptable salt form, e.g., ketotifen fumarate.

In still another aspect, the invention provides any of methods I-VI as follows:
2.1 Any of methods I-VI, wherein the effective amount of mast cell stabilizer is effective to inhibit anaphylaxis;
2.2 Any of methods I-VI or 2.1, wherein the mast cell stabilizer is selected from a group consisting of: cromolyn, cromoglycate, nedocromil, bepotastine, ebastine, epinastine, azelastine, lodoxamide tromethamine, lodoxamide trometamol, pemirolast, olopatadine, ketotifen, norketotifen, 10-H-norketotifen, 9-OH-norketotifen, 9,10-di-OH-norketotifen and those having the general formula:

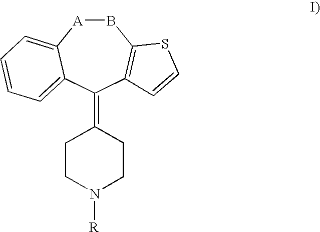

wherein:
a) A-B— is a moiety having the formula:
   i) —CO—CH₂
   ii) —CH₂—CO—
   vii) —CO—CO—; and
b) R is a hydroxyalkyl or a carboxyalkyloxyalkyl moiety;

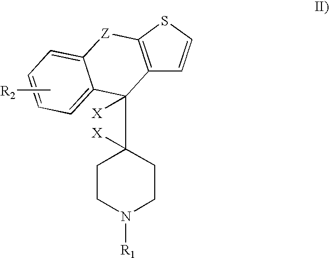

wherein:
a) —Z— is —CH₂—CH₂— or —CH═CH—;
b) R₁ is a H or C₁₋₄alkyl; and
c) R₂ is H or halogen.

or

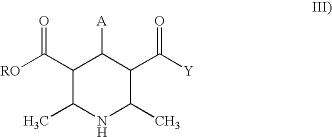

wherein:
a) A is

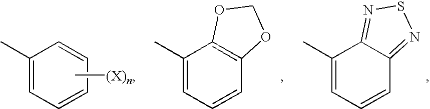

b) n is 1 or 2;
   when n is 1, X is H, halogen, CF₃, alkyne, —S(O)_{n″}R, OR or NO₂;
   when n is 2, X is H or halogen;
c) n′ is 0, 1 or 2;
d) R is C₁₋₅(un)branched alkyl;
e) Y is Z(C(R′)(R′))_{n′}Z′ or

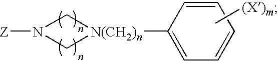

wherein:
i) n″ is 2-4;
ii) m is 0, 1, 2, 3;
iii) R′ is H or R;
iv) X′ is H, halogen, CF₃, alkyne, —S(O)_{n′}R, OR or NO₂;
v) Z is NH or O;
vi) Z′ is N(R′)(R″) or

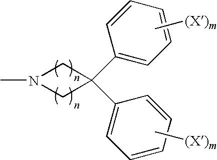

or R″ is

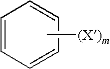

in free or pharmaceutically acceptable acid addition salt form;
2.3 Any of the preceding methods wherein the allergen is administered via the oral route;
2.4 Any of the preceding methods wherein the allergen is administered via injection;
2.5 Any of the preceding methods wherein the allergen is administered via inhalation;
2.6 Any of the preceding methods wherein the allergen is administered sublingually;
2.7 Any of the preceding methods wherein the allergen is food allergen;
2.8 Any of the preceding methods wherein the allergen is peanut allergen;
2.9 Any of the preceding methods wherein the allergen is airborne allergen;
2.10 Method 2.9, wherein the airborne allergen is plant allergen;
2.11 Method 2.9, wherein the airborne allergen is animal dander;
2.12 Method 2.9, wherein the airborne allergen is dust mites;
2.13 Any of the preceding methods, wherein the airborne allergen is selected from a group consisting of grass pollen, weeds, timothy grass, ragweed, birth tree pollen, grass pollen, mold, fungus spores, dust and animal dander;
2.14 Any of methods I-VI or 2.1-2.13, wherein the allergen is administered in increasing dosages over a shorter period of time than when the allergen is administered in the absence of the administration of a mast cell stabilizer;
2.15 Any of methods I-VI or 2.1-2.13, wherein the allergen is administered at a higher dosage than the dosage of allergen administered in the absence of a mast cell stabilizer;
2.16 Any of methods I-VI or 2.1-2.15, wherein the allergen is administered at higher frequency than the frequency of the allergen that is administered in the absence of a mast cell stabilizer;
2.17 Any of methods I-VI or 2.1-2.16, wherein the allergen is administered at higher dosage and/or frequency than the frequency and dosage of the allergen that is administered in the absence of a mast cell stabilizer;
2.18 Any of methods I-VI or 2.1-2.17, wherein the mast cell stabilizer is administered prior to the commencement of each dose of allergen;
2.19 Any of methods I-VI or 2.1-2.18, wherein the mast cell stabilizer is administered up to two weeks prior to commencement of each does of allergen;
2.20 Any of methods I-VI or 2.1-2.19, wherein the mast cell stabilizer is administered up to 24 hours prior to commencement of each dose of allergen;
2.21 Any of methods I-VI or 2.1-2.20, wherein the mast cell stabilizer is ketotifen in free or pharmaceutically acceptable acid addition salt form;
2.22 Any of methods I-VI or 2.1-2.21, wherein the pharmaceutically acceptable salt is selected from a group consisting of hydrochloride, hydrobromide, hydrogen sulfate, phosphate, maleate, nitrate, besylate and fumarate;
2.23 Any of methods I-VI or 2.1-2.22, wherein the pharmaceutically acceptable salt is fumarate salt;
2.24 Any of methods I-VI or 2.1-2.23, wherein the mast cell stabilizer is ketotifen fumarate;
2.25 Any of methods I-VI or 2.1-2.24, wherein the mast cell stabilizer is cromolyn;
2.26 Any of methods I-VI or 2.1-2.25, wherein the cromolyn is administered in combination with one or more leukotriene modifier;
2.27 Any of methods I-VI or 2.1-2.26, wherein the leukotriene modifier is Zileuton or Montelukast;
2.28 Any of methods I-VI or 2.1-2.27, wherein further comprises administering said ketotifen in free or pharmaceutically acceptable acid addition salt thereof in conjunction with one or more therapeutic agents selected from a group consisting of histamine-1 receptor antagonists, histamine-2 receptor antagonists, corticosteroids, PDE IV inhibitors, beta-adrenergic agonists, anticholinergic agents, epinephrine, IgE antagonists and antagonists of cytokines, kinase inhibitors, and inhibitors of kinase-activated pathways, in free or pharmaceutically acceptable acid addition salt form.

### DETAILED DESCRIPTION OF THE INVENTION

The present invention relates to the prophylactic treatment, delay, control and/or reduction of severe reaction to food (e.g., peanut, tree nuts), hymenoptera venom (e.g., bee venom), drugs (e.g., β-lactam antibiotics, insulin (e.g., Humulin, Novolin), streptokinase, allergen extracts), and/or airborne allergen such as plant allergen (e.g., grass pollen, weed, ragweed, tree pollen, mold), dust and/or animal or pet dander, in patients at risk of systemic anaphylaxis. In addition, the current invention also relates to any of the foregoing methods, Method I, II, II-A, III, IV, wherein the allergen is dust mite.

The terms “antigen” and “allergen” may be used interchangeably herein.

As used herein, the term “anaphylaxis” or “anaphylactic reaction” refers to a Type I hypersensitive reaction, i.e., a rapid-onset hypersensitive IgE-mediated reaction to antigen(s) or allergen(s). The initial manifestation of reaction may range from urticaria (hives), pruritis (itching), allergic rhinitis (hay fever), mild asthma to brochospasm or mucosal edema as well as hypotension and/or syncope. Symptoms of anaphylaxis in cases of food or hymenoptera allergy are typically manifested relatively quickly, within five to thirty minutes of exposure, rarely more than two hours. Patients at risk of anaphylaxis include those who have been exposed to specific antigen(s) or allergen(s), but did not develop tolerance to it and will likely have a hypersensitive reaction upon re-exposure. Antigen(s) or allergen(s) that typically cause anaphylaxis include dust mites, plant pollens, hymenoptera venom (i.e. bee, wasp, hornet, yellow jacket, fire ants) certain drugs (i.e. penicillin) and various food products (i.e. seafood, tree nuts, seeds, soy bean, milk, peanuts and eggs). In the case of peanut allergen, hypersensitivity may be so severe that prior exposure of trace amount (such as inhaling peanut dust or ingesting it through breast milk) may be unknown to the individual, rendering a hypersensitivity to peanut on a seemingly initial exposure.

The term “local anaphylaxis” refers to a hypersensitive IgE-mediated reaction to antigen(s) or allergen(s) that results in moderate to severe swelling, redness and/or itching within a limited area of the body, typically near the site of exposure. Typical manifestation of local anaphylaxis includes allergic rhinitis, pruritis, mild asthma and/or urticaria.

The term “systemic anaphylaxis” or “systemic anaphylactic reaction” refers to a systemic hypersensitive reaction to antigen(s) or allergen(s) that results in manifestation of at least one of the symptoms including, but not limited to: difficulty breathing or swallowing as a result of swelling of the tongue, lips and/or oropharynx; wheezing as a result of bronchospasm or mucosal edema; light-headedness; hypotension; syncope; tachycardia; shock; and/or cardiac arrest. Antigen(s) or allergen(s) that typically causes systemic anaphylaxis include certain medicaments (such as penicillin), latex, hymenoptera venom (such as been, wasp, fire ant, yellow jacket and hornet venom) and food allergens (such as eggs, milk, soy, fish, shellfish and various types of fruits, seeds and nuts). Other antigen(s) or allergen(s) including, but not limited to allergens such as pollen (e.g., ragweed extracts) typically does not cause systemic anaphylaxis, but may cause systemic anaphylaxis due to the patient's high exposure to the allergen, e.g., high or frequent dosages administered during allergy desensitization therapy, e.g., intentionally or unintentionally rushed desensitization therapy. High dosages or high frequency of administration of the allergen may vary from patient to patient depending the level of sensitivity of that patient to the allergen.

The term “severe reaction” refers to systemic anaphylactic reaction.

The term “symptoms of mediator release” refers to an onset of a local or systemic anaphylactic reaction. Symptoms of mediator release may include, but not limited to urticaria, intense itching (pruritis), decrease in blood pressure, angioedema (e.g., swelling of the mouth and lips), mild asthma and/or gastro-intestinal side effects such as vomiting or diarrhea. Other gastro-intestinal side effects may include nausea, abdominal pain, vomiting, diarrhea and colic.

Patients “at risk of systemic anaphylaxis” typically include those who have had one serious reaction involving the airway or cardiovascular systems upon initial exposure to antigen(s) or allergen(s) such as food allergen (e.g. shellfish, peanuts, tree nuts, milk, eggs, poppy seeds, Kiwi, or their components, e.g., casein, lactalbumin and lactaglobulin in milk/dairy products, and tropomyosin in shellfish), particularly peanut allergen, and hymenoptera venom allergen (e.g. bee, yellow jacket, hornet and wasp), particularly bee venom allergen. This patient group also includes those who have a history of food allergy in addition to a precondition of asthma or a history of wheezing during respiratory illnesses in the absence of asthma. Children with food allergy are also at risk of systemic anaphylaxis if other family members have experienced systemic anaphylactic reaction. Patients that are particularly vulnerable to systemic anaphylaxis also include those who regularly use β-blockers (e.g. Propanolol). Studies have shown that β-blockers competitively inhibit catecholamine (epinephrine) from binding to β-adrenergic receptor cites. Because catecolamine (epinephrine) induces smooth muscle relaxation and brochodilation by stimulation of β-receptors, competitive binding of β-blockers to β-receptor cites therefore tends to reduce the effectiveness of epinephrine. Since epinephrine is the number one choice of emergency treatment of anaphylaxis, patients who are regular users of β-blockers are therefore particularly vulnerable as a result of epinephrine resistance.

Patients who are “regular users of β-blockers” herein refer to individuals who take either selective or non-selective β-adrenergic antagonist agents such as propranolol, on a regular basis. Patients who are “regular users of β-agonist” herein refer to individuals who take β₂-adrenergic agonist agents such as albuterol, on a regular basis, especially patients who take long acting β₂-adrenergic agonist agents such as formoterol or salmeterol, particularly over a sustained period, e.g., at least four weeks.

Patients having a “B16-Arg/Arg genotype” (also refer to as B16-Arg/Arg patient) herein refer to individuals who have homozygous alleles encoding arginine (Arg) at the 16 position of β₂-adrenergic receptors. These patients appear to be particularly susceptible to deterioration in pulmonary function as a result of regular use of β₂-adrenergic agents. See, Israel, et al., Am J Respir Crit. Care Med, Vol 162. pp 75-80 (2000). Genomic DNA may be prepared for genotopic analysis by standard techniques. See Maniatis, et al., Molecular Cloning: A Laboratory Manual. Cold Spring Harbor Laboratory, New York (1989). Analysis of B16 genotype may be done, e.g., by amplification refractory mutation system (ARMS).

The phrase “epinephrine resistance” refers to patients who exhibit a deterioration in pulmonary function as a result of regular use of either β-blockers or β-agonists.

“Deterioration in pulmonary function” refers to a decline in peak expiratory flow of more than 20 L/min.

The phrase “elevated antigen-specific IgE” level herein refers to a level of antigen-specific IgE greater than that in patients with no hypersensitivity to such antigen(s) or allergen(s). The level of antigen-specific IgE is measured by the radioallergosorbent test (RAST), whereby polymer-conjugated allergen is mixed with the blood serum to be tested. Allergen-specific IgE is then quantified by adding ¹²⁵I labeled anti-IgE antibody and measuring the amount of radioactivity of the conjugate. The level of antigen-specific IgE can also be measured by fluorescent enzyme immunoassay well known in the art and commercially available (e.g. ImmunoCAP®, or CAP-System, FEIA, Pharmacia Upjohn). Patients with no hypersensitivity to peanuts typically maintain an antigen-specific IgE level of less than 30 kU/L, while patients at risk of systemic anaphylaxis typically has a history of sensitivity to peanut and peanut-specific IgE level of 30 kU/L.

The phrase “elevated basal serum tryptase level” herein refers to total mast-cell specific enzymes, which include alpha-tryptase and beta-tryptase. These enzymes are released during acute allergic reactions, indicating mast cell activation and can be measured by using methods well known in the art and commercially available (e.g. Uni-CAP-Tryptase fluoroimmunoassay, Pharmacia & Upjohn, Uppsala, Sweden).

The term “skin prick test” (SPT) refers to an allergy test that involves placing a drop of dilute allergen extract to be tested onto the skin on the forearm or the back of the patient. A small needle is used to prick the skin below the drop of allergen. The site is examined over the next twenty minutes for evidence of swelling and redness. A drop of saline and a drop of histamine solutions (10 mg/mL) are also tested as a negative and positive control, respectively. The wheal diameter is then measured in millimeters. Patients who are severely allergic to peanuts have a positive SPT, wherein the mean wheal diameter is typically greater than 8 mm. Children who are under one year of age, e.g., infants or newborns, and are allergic to peanut typically have a mean wheal diameter of 4.5 mm from the SPT.

The term “hymenoptera allergen” refers to allergen from the venom of a member of the order Hymenoptera, e.g. bee, wasp, yellow jacket, hornet or fire ant venom, especially bee venom. Hymenoptera allergen may also include abstract or extract of the venom, a specific allergen protein, mixture of allergen proteins, genetically manufactured allergen, or any combinations thereof.

The term “food allergen” refers to allergen from food, particularly from eggs, milk, soy, fish, shellfish and various types of fruits, seeds and nuts, most especially from peanuts and tree nuts. Food allergen may also include abstract or extract of the food, e.g., eggs, fish, fruits, etc., a specific allergen protein, mixture of allergen proteins, genetically manufactured allergen, or any combinations thereof.

The term “infant” refers to any one who is one month to two years of age. The term “a child” herein generally refers to anyone who is two to twelve years of age unless specify otherwise. The term “adolescent” herein refers to anyone who is twelve to twenty-one years of age.

The term “airborne allergen” refers to, for example, plant matters (e.g., grass pollen, weeds, timothy grass, ragweed, birth tree pollen, grass pollen, mold, fungus spores, etc), and other airborne particles such as dust and/or animal dander (e.g., skin flakes, saliva, urine, hair, droppings, etc. from animals such as birds, cats, dogs, mice, rats, cockroaches, hamsters, rabbits, guinea pigs, horses, cows, fish, etc.). In another embodiment, airborne allergen may include dust mites. Airborne allergen may also include abstract or extract of the allergen, e.g., pollen, dust mites, mold, fungi, animal dander, etc., a specific allergen protein, mixture of allergen proteins, genetically manufactured allergen, or any combinations thereof.

The term “desensitization” treatment refers to an immunotherapy whereby small doses of specific antigen or allergen are administered to a patient over a period of time so as to develop a tolerance for the antigen or allergen. Preferably the dose is increased over the period of time. The dose of the antigen or allergen to be administered, and the period of time required to develop tolerance for the antigen or allergen can be determined by one skilled in the art. The antigen or allergen may be administered to the patient through any route known in the art, including, but not limited to oral, inhalation, epicutaneous, intranasal, rectal, and parenteral routes (intravenous, intramuscular, subcutaneously, and intraperitoneal). The allergen or antigen may also be administered sublingually, e.g., via the sublingual or sublingual and spit method. In a particular embodiment, desensitization comprises administering one or more specific antigens or allergens so as to induce tolerance for such antigens or allergens.

With the administration of the mast cell stabilizers of the invention, e.g., ketotifen, to prevent or inhibit mediator release, the allergen(s) may be administered at higher dosages and/or at higher frequency than when the allergen is administered in the absence of a mast cell stabilizer. Therefore, the desensitization process may be shortened or expedited (i.e., RUSH immunotherapy). For example, a patient undergoing desensitization may begin with RUSH immunotherapy (i.e., expedited desensitization) wherein said patient receives on day one a series of small, escalating doses of allergen in conjunction with (e.g., during, before or after the administration of) the mast cell stabilizer to inhibit or prevent mediator release. Said patient then receives increasing doses of allergen at a slower frequency so as to build-up to a maintenance dose over a period of time. The maintenance dosage and the period of time during which the maintenance doses are administered may vary depending on each patient and said patient's sensitivity to the allergen and may last for years or even indefinitely. By undergoing RUSH immunotherapy with the use of the mast cell stabilizer, however, said patient may be able tolerate larger and/or more frequent doses of allergen with fewer adverse events, thereby, improving efficacy, enhancing patient compliance with desensitization protocol, and reducing the number of doctor's office visits. Therefore, during the initial administration of the allergen, the administration may be carried out more frequently, e.g., every thirty minutes, every hour, etc., at increasingly higher doses instead of administering the allergen over a longer period of time due to adverse reaction to the allergen in the absence of the administration of a mast cell stabilizer, e.g., ketotifen to prevent or inhibit mediator release. The frequency and dosages of the allergen may vary from patient to patient depending on the level of sensitivity of patient to a particular allergen. Such frequency and dosage, however, may be determined by one skilled in the art.

The term “mast cell stabilizer” refers to any compound or agent capable of inhibiting or reducing the release of inflammatory mediators or other autacoids such as histamine, phospholipase A₂, platelet-activating factor, metabolites of arachidonic acid, leukotriene D₄, and kinins. Examples of mast cell stabilizers include, but are not limited to cromolyn or cromoglycate, nedocromil, bepotastine, ebastine, epinastine, azelastine, lodoxamide tromethamine, lodoxamide trometamol, pemirolast, olopatadine, ketotifen and their derivatives, e.g., ketotifen metabolites, e.g., norketotifen (i.e., 4-(4-piperidinylidene)-4H-benzo[4,5]cyclohepta[1,2-b]thiophen-10(9H)-one), 10-H-norketotifen, 9-OH-norketotifen, 9,10-di-OH-norketotifen as disclosed in U.S. Pat. No. 6,207,684, herein incorporated by reference. The terms “10-OH-norketotifen”, “9-OH-norketotifen” and “9,10-di-OH-norketotifen” refer to norketotifen analogs wherein the hydroxy group(s) is substituted on the 9^{th} and/or 10^{th} position of the 7-membered ring of norketofen in place of oxo. In addition to these ketotifen derivatives, the mast cell stabilizers of the present invention also include other derivatives having the general formula:

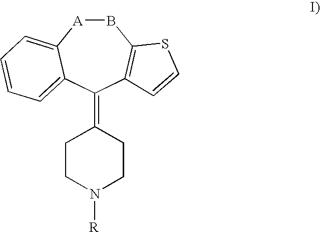

in free or salt form wherein:
a) -A-B— is a moiety having the formula:
   i) —CO—CH₂
   ii) —CH₂—CO—
   iii) —CH₂—CH₂—
   iv) —CHOH—CH₂—
   v) —CHOH—CHOH—
   vi) —CH₂—CHOH—; or
   vii) —CO—CO—; and
b) R is a hydroxyalkyl or a carboxyalkyloxyalkyl moiety;

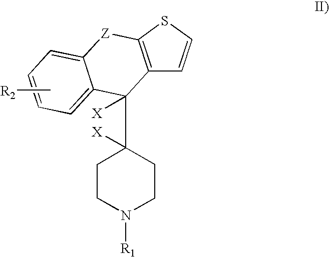

in free or salt form wherein:
a) —Z— is —CH₂—CH₂— or —CH═CH—;
b) R₁ is a H or C₁₋₄alkyl; and
c) R₂ is H or halogen.

or

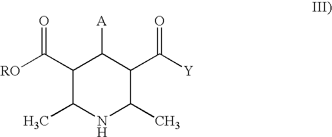

in free or salt form wherein:
a) A is

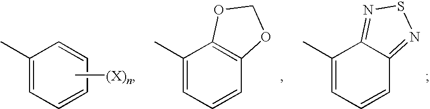

b) n is 1 or 2;
   when n is 1, X is H, halogen, CF₃, alkyne, —S(O)_{n′}R, OR or NO₂;
   when n is 2, X is H or halogen;
c) n′ is 0, 1 or 2;
d) R is C₁₋₅(un)branched alkyl;
e) Y is Z(C(R′)(R′))_{n″}Z′ or

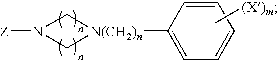

wherein:
i) n″ is 2-4;
ii) m is 0, 1, 2, 3;
iii) R′ is H or R;
iv) X′ is H, halogen, CF₃, alkyne, —S(O)_{n′}R, OR or NO₂;
v) Z is NH or O;
vi) Z′ is N(R′)(R″) or

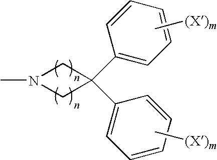

or R″ is

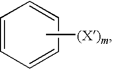

in free or pharmaceutically acceptable acid addition salt form, the subject matter of which are disclosed in U.S. Pat. Nos. 6,207,683, 3,491,103 and 3,682,930, which all are herein incorporated by reference. Mast cell stabilizer also includes ketotifen in free or pharmaceutically acceptable salt thereof. Preferably, the mast cell stabilizer of the present invention is ketotifen, most preferably, ketotifen fumarate.

Ketotifen is known in the art, and may be made via known methods in the art (U.S. Pat. No. 3,682,930; U.S. Pat. No. 3,272,826) or obtained commercially. Methods for preparing ketotifen derivatives as described above (e.g., norketotifen, 10-OH-norketotifen, etc.) are disclosed in U.S. Pat. Nos. 6,207,683, 6,207,684, 3,491,103, 3,682,930 and Waldvogel et al., Helv Chem Acta (1976) 59:866-877, the contents of each of which are herein incorporate by reference.

Pharmaceutically acceptable acid addition salts of ketotifen may be useful in the present invention, which include, but are not limited to hydrochloride, hydrobromide, hydrogen sulfate, phosphate, nitrate, besylate, maleate and fumarate salts, especially the fumarate salt.

“An effective amount” refers to any amount that is effective to bring about a desirable effect. Therefore, an effective amount of ketotifen or mast cell stabilizer to inhibit and/or prevent symptoms of mediator release refers to an amount sufficient to inhibit and/or prevent symptoms of mediator release. An effective amount may vary for each patient depending on the severity of the allergic response and the age and weight of that patient. Determination of such amount is well within the knowledge of those skilled in the art.

Mast cell stabilizers may be administered orally, parenterally, transmucosally or transdermally as known in the art. A typical dosage, i.e., of ketotifen may be administered up to 8 mg twice daily. A typical dosage may include oral administration of 0.5-2 mg twice daily for adults and children 3 years of age and older, e.g. 1 mg b.i.d. A typical dosage for infants and children from 6 months to 3 years of age may be 0.05 mg per kilogram of body weight twice daily, once in the morning and one in the evening. Unless otherwise noted, dosages of mast cell stabilizers as set forth herein are by weight equivalent to the free base irrespective of whether the drug is administered in salt form or not. The mast cell stabilizers may be administered alone, in conjunction or simultaneously with other therapeutic agents. These therapeutic agents include but are not limited to histamine-1 receptor antagonists, histamine-2 receptor antagonists, corticosteroids, PDE IV inhibitors, beta-adrenergic agonists, anticholinergic agents, epinephrine and IgE antagonists (including variant anti-IgE antibodies (non-human mammalian, chimeric, polyclonal and/or monoclonal antibodies (Mabs) and fragments thereof (e.g., proteolytic digestion or fusion protein products) and nucleotides or polypeptides encoding or containing IgE sequences or nucleotides or polypeptides that induce anti-IgE antibody production (e.g., anti-IgE immunogenic polypeptide as disclosed in U.S. Pat. No. 6,913,749, U.S. Pat. App. No. 2004/0076625A1 and 2003/0031663A1), especially IgE antagonist and polypeptides capable of differential binding to Fc.epsilon.RI and Fc.epsilon.RII.); antagonist of cytokines, such as, for example, monoclonal antibody to TNF-alpha, soluble TNF-alpha receptor-Fc fusion protein, and/or IL-1 receptor; kinase inhibitors, e.g., inhibitors of spleen tyrosine kinase (syk), and inhibitors of kinase-activated pathways, e.g. inhibitors of STAT proteins. Therefore, ketotifen may be administered concomitantly, e.g., as an admixture; separately but simultaneously or concurrently; or sequentially with therapeutic agents as disclosed herewith.

With the method of desensitization, ketotifen may be administered prior to, at the same time or after administration of each dose of allergen. The mast cell stabilizer may be administered 2 weeks prior to the administration of each dose of allergen. In one embodiment, the mast cell stabilizer is administered 24 hours prior to each dose of allergen. More preferably, the mast cell stabilizer is administered orally. In an especially preferred embodiment, the mast cell stabilizer is ketotifen fumarate and is administered orally.

For example, in one embodiment, ketotifen is administered to an adult undergoing allergy desensitization therapy, orally in an amount of 2.25-4.5 mg b.i.d. for a period of 1-9 months, e.g., 3 months, from start of desensitization therapy.

Similarly, the allergen may be administered via any method known in the art. Therefore, the allergen may be administered either in the form of an extract, e.g., pollen extract, peanut extract or in the form of solid or powder, e.g., via inhalation, orally, subcutaneously, sublingually, e.g., sublingual oral (SLIT) or sublingual spit (SPIT) method. In normal desensitization process, the allergen is administered at a small dosage initially and more frequently and then slowly increases to a higher dosage until a maintenance dosage is reached. With RUSH or expedited immunotherapy, the administration of the allergen may be carried out at a higher dosages and frequency than when the dosage and frequency where the allergen is administered in the absence of the administration of a mast cell stabilizer, e.g., ketotifen. The patient undergoing RUSH immunotherapy therefore has the benefit of minimizing adverse reactions to the allergen, therefore enhancing patient compliance and efficacy with the desensitization protocol. The frequency and dosage of the allergen may vary from patient to patient depending on the level of sensitivity of patient to a particular allergen. Such frequency and dosages may be determined by one skilled in the art.

### Example 1

### The Use of Ketotifen in Patients with Peanut Allergy

To analyze hypersensitivity to an allergen, e.g., peanut, a double-blind, randomized, dose-ranging study is carried out with patients with a history of peanut allergy manifested by urticaria, angioedema, respiratory tract symptoms or hypotension generally using the methods described in Israel et al., Am. J. Respir. Crit. Care Med., (2000) 164:75-80. Eligible patients include those with serum total IgE level of 30-1000 IU/mL and/or a positive skin-prick test to peanut. Patients must have asthma condition under control with a forced expiratory volume in one second that is at least 80 percent of the predicted value. Patients may not have taken systemic corticosteroids, beta-blockers, acetylcholinesterase, aspirin, antihistamines and anti-depressants prior to and throughout the study.

Before enrollment, threshold level for reactivity to peanut allergen is confirmed by a screening double-blind, placebo-controlled oral food challenge. Base line spirometry, peak expiratory flow rates and continuous cardiac monitoring is established. Vital signs are checked, chest auscultation is performed, and peak expiratory flow rates are monitored every 30 minutes during the food challenge and for at least 2 hours after the last dose or the abatement of any symptoms or signs. Patients are given an initial dosage of 1 mg of peanut followed successively by 5, 10, 20, 50, 100, 200, 500, 1000 and 2000 mg of peanut flour or matching placebo capsules every 40 minutes until a definite reaction is occurring, such as manifestation of nausea, abdominal pain, vomiting, throat tightness, chest tightness, wheezing, persistent cough, rhinitis, conjunctivitis, pruitus, hives, and angioedema. To maximize safety and prevent severe reactions, the end point for the oral food challenge is the threshold dose for an allergic reaction.

Peak expiratory flow rates are monitored every 30 minutes during food challenge for at least 2 hours after the last does or abatement of any symptoms or signs. A tolerance of 2000 mg of peanut flour is considered to have a negative allergy test result. If patients manifest clear signs of hypersensitivity reaction, the patient is then given activated charcoal slurry (Liqui-Char, Jones Pharma) to adsorb residual peanut protein in the stomach. Each patient qualified to enter the study is required to have one positive and one negative result to peanut at screening.

Peanut flour is made by grinding equal portions of Valencia, runner, and Spanish peanuts (Greer Laboratories. The peanuts are defatted, and then various doses (1 mg to 2 g) are loaded into gel capsules. Matching placebo capsules are filled with similar amounts of cornstarch. For masking purposes, the capsules are rolled in tuna oil before administration.

The double-blind, randomized study is carried out in two groups with test patients receiving 1 mg of ketotifen or placebo orally twice daily, once in the morning and once in the evening. Patients then undergo an oral food challenge with peanut flour at two, four and six weeks. The oral food challenge with peanut flour alone is initiated at 1 mg or 100 mg, depending on the threshold determined during the screening study, and is then escalated in accordance with the above schedule to 4000 mg and then 8000 mg if tolerated. Each dose level is to be completed before enrollment at the next level. Every two weeks, blood and urine samples are obtained. Total peanut-specific IgE level is measured by fluorescence enzyme immunoassay (CAP-System, FEIA).

The predefined primary efficacy measure is the change from base line in the log-transformed threshold dose of peanut flour that induced hypersensitivity. The ketotifen should significantly increase the patients' threshold sensitivity to peanut upon oral ingestion, and so be expected to reduce the likelihood and/or severity of anaphylactic reaction in the event of future exposure to peanuts.

### Example 2

### The Use of Ketotifen in Patients with Ragweed (Airborne) Allergy

The use of ketotifen in patients with airborne allergen, e.g., pollen, ragweed, etc., may be carried out similar to Example 1 except airborne allergen extract, e.g., pollen extract or ragweed extract, is used instead of oral peanut allergen.

### Example 3

### The Use of Ketotifen in Patients Undergoing Allergen Desensitization for Peanut Allergy

To analyze hypersensitivity to peanut, a double-blind, randomized, dose-ranging study is carried out with patients with a history of peanut allergy manifested by urticaria, angioedema, respiratory tract symptoms or hypotension generally using the methods described in Israel et al., Am. J. Respir. Crit. Care Med., (2000) 164:75-80. Eligible patients include those with serum total IgE level of 30-1000 IU/mL and/or a positive skin-prick test to peanut. Patients must have asthma condition under control with a forced expiratory volume in one second that is at least 80 percent of the predicted value. Patients may not take systemic corticosteroids, beta-blockers, acetylcholinesterase, aspirin, antihistamines and anti-depressants during the study.

Before enrollment, threshold level for reactivity to peanut allergen is confirmed by a screening double-blind, placebo-controlled oral food challenge. Base line spirometry, peak expiratory flow rates and continuous cardiac monitoring is established. Vital signs are checked, chest auscultation is performed, and peak expiratory flow rates are monitored every 30 minutes during the food challenge and for at least 2 hours after the last dose or the abatement of any symptoms or signs. Patients are given an initial dosage of 1 mg of peanut followed successively by 5, 10, 20, 50, 100, 200, 500, 1000 and 2000 mg of peanut flour or matching placebo capsules every 40 minutes until a definite reaction is occurring, such as manifestation of nausea, abdominal pain, vomiting, throat tightness, chest tightness, wheezing, persistent cough, rhinitis, conjunctivitis, pruitus, hives, and angioedema. To maximize safety and prevent severe reactions, the end point for the oral food challenge is the threshold dose for an allergic reaction.

Peak expiratory flow rates are monitored every 30 minutes during food challenge for at least 2 hours after the last does or abatement of any symptoms or signs. A tolerance of 2000 mg of peanut flour is considered to have a negative allergy test result. If patients manifest clear signs of hypersensitivity reaction, the patient is then given activated charcoal slurry (Liqui-Char, Jones Pharma) to adsorb residual peanut protein in the stomach. Each patient qualified to enter the study is required to have one positive and one negative result to peanut at screening.

Peanut flour is made by grinding equal portions of Valencia, runner, and Spanish peanuts (Greer Laboratories. The peanuts are defatted, and then various doses (1 mg to 2 g) are loaded into gel capsules. Matching placebo capsules are filled with similar amounts of cornstarch. For masking purposes, the capsules are rolled in tuna oil before

Peanut capsules, placebo and Ketotifen tablet are administered orally.

The double-blind, randomized study is carried out in four groups with test patients receiving placebo or 0.5, 1, or 2 mg of ketotifen orally twice daily, once in the morning and once in the evening. Patients then undergo an oral food challenge with peanut flour at two, four and six weeks. The oral food challenge with peanut flour alone is initiated at 1 mg or 100 mg, depending on the threshold determined during the screening study, and is then escalated in accordance with the above schedule to 4000 mg and then 8000 mg if tolerated. Each dose level is to be completed before enrollment at the next level. Every two weeks, blood and urine samples are obtained. Total peanut-specific IgE level is measured by fluorescence enzyme immunoassay (CAP-System, FEIA).

The predefined primary efficacy measure is the change from base line in the log-transformed threshold dose of peanut flour that induced hypersensitivity. The ketotifen should significantly increase the patients' tolerance to peanut upon oral ingestion, and so be expected to reduce the likelihood and/or severity of anaphylactic reaction in the event of future exposure to peanuts.

### Example 4

### The Use of Ketotifen in Patients Undergoing Allergen Desensitization for Bee Venom Allergy

To analyze hypersensitivity to bee venom, a double-blind, randomized, dose-ranging study is carried out with patients with a history of bee sting allergy manifested by urticaria, angioedema, respiratory tract symptoms or hypotension generally using the methods described in Israel et al., Am. J. Respir. Crit. Care Med., (2000) 164:75-80. Eligible patients include those with serum total IgE level of 30-1000 IU/mL and/or a positive skin-prick test to bee venom. Patients must have asthma condition under control with a forced expiratory volume in one second that is at least 80 percent of the predicted value. Patients may not take systemic corticosteroids, beta-blockers, acetylcholinesterase, aspirin, antihistamines and anti-depressants during the study.

Before enrollment, threshold level for reactivity to bee venom allergen is confirmed by a screening double-blind, placebo-controlled bee venom challenge. Base line spirometry, peak expiratory flow rates, bee venom specific IgE level, basal tryptase level, and continuous cardiac monitoring are established. Vital signs are checked, chest auscultation is performed, and peak expiratory flow rates are monitored and bee venom specific IgE level and basal tryptase level are measured every 30 minutes during the challenge and for at least 2 hours after the last dose or the abatement of any symptoms or signs. Intradermal skin tests are performed on the surface of forearm using purified and immunochemically characterized bee venom at concentration of 0.01, 0.1, 1, 10, or 100 μg/ml or matching placebo (histamine) every 40 minutes until a definite reaction is occurring. The wheal size is evaluated 15 min after application. A wheal of 3 mm or greater is considered appositive reaction. Patients are closely monitored for any systemic reaction. Each patient qualified to enter the study is required to have one positive and one negative result to bee venom at screening.

Both placebo and ketotifen tablet are administered orally.

The double-blind, randomized study is carried out in four groups with test patients receiving placebo or 0.5, 1 or 2 mg of ketotifen orally twice daily, once in the morning and once in the evening. Patients then undergo a challenge with bee venom at two, four and six weeks. The challenge with pure bee venom alone is initiated at 0.01 μg, depending on the threshold determined during the screening study, and is then escalated in accordance with the above schedule to 100 μg/ml and then if tolerated, 200 μg/ml. Each dose level is to be completed before enrollment at the next level. Every two weeks, blood and urine samples are obtained. Total bee venom-specific IgE level is measured by fluorescence enzyme immunoassay (CAP-System, FEIA).

The predefined primary efficacy measure is the change from base line in the log-transformed threshold dose of bee venom that induced hypersensitivity. The ketotifen should significantly increase the patients' tolerance to bee venom, and so be expected to reduce the likelihood and/or severity of anaphylactic reaction in the event of future exposure to bee sting.

### Example 5

### The Use of Ketotifen in Patients with Peanut Allergy Undergoing Oral Peanut Allergen Desensitization Therapy

A study is performed to evaluate the dose-related effects of ketotifen fumarate on the scope and severity of allergic signs and symptoms resulting from concomitant oral peanut tolerization therapy. The study also evaluates the effects of discontinuation of ketotifen therapy on the scope and severity of allergic signs and symptoms resulting from concomitant oral peanut tolerization therapy after 12 or 16 weeks, while oral peanut tolerization therapy is continued. The study also evaluates the effects of ketotifen therapy on the scope and severity of allergic signs and symptoms resulting from concomitant oral peanut tolerization therapy when ketotifen dosing is initiated after 12 weeks of oral peanut tolerization therapy. The study generally consists of screening, baseline, treatment, and post-treatment follow-up periods, and is randomized, double-blind, placebo-controlled, and in parallel groups.

Patients selected are generally healthy subjects between the ages of 2 and 50 with an allergy to peanut which provokes signs and symptoms of an anaphylactic reaction. The patients may also have co-morbid atopic dermatitis and/or allergic rhinitis. If a patient is selected for the study, they are randomly assigned a treatment of 0.5 mg oral ketotifen fumarate, 2.0 mg oral ketotifen fumarate, or placebo (n=12 for each group).

During the treatment period, patients receive ketotifen fumarate or placebo for one week prior to tolerization therapy. At week 1, oral peanut tolerization therapy is begun with peanut flour, and safety evaluations are performed. The oral dose of peanut flour is increased at weeks 3, 5, 7, 9, 11 and 13, and safety evaluations are performed at that time.

At week 13, half of the patients in each of the ketotifen groups are switched in a double-blind fashion to placebo, and subjects in the placebo treatment group are switched in a double-blind fashion to receive 2.0 mg oral ketotifen. Patients continue in the study for an additional 4 weeks. At weeks 15 and 17, the peanut flour dose is increased, and safety evaluations are performed. During the last 4 weeks, escape therapy (2 mg ketotifen) is available should any allergic reactions occur.

Patients return for post-treatment follow-up safety evaluations approximately 4 weeks after their last dose of study medication is ingested. The peanut tolerization therapy may be continued for up to 9 months.

The measure of this study is the amount of peanut flour that is well-tolerated. Changes in co-morbid related allergies are documented. During the final 4 weeks of the treatment period, the number of patients requiring escape therapy is also compared between treatment groups.

It is expected that patients receiving ketotifen will be able to tolerate more peanut flour than patients in the placebo group. It is also expected that patients initially receiving ketotifen and then switched to placebo at week 13 will require less escape therapy than patients continuously receiving the placebo. It may also be found that patients initially receiving ketotifen and then switched to placebo at week 13 will require similar escape therapy as patients continuously receiving ketotifen.

The matters set here are offered by way of illustration only and not as limitations. While particular embodiments have been shown and described, it will be apparent to those skilled in the art that changes and modifications may be made without departing from the broader aspects of invention.

1. A method for prophylactically treating, controlling, delaying or reducing severe reaction to allergen or symptoms of mediator release during allergen desensitization in a patient at risk of systemic anaphylaxis if exposed to such allergen, comprising: administering to said patient an effective amount of ketotifen in free or pharmaceutically acceptable acid addition salt.

2. The method according to claim 1, wherein said acid addition salt is selected from the group consisting of hydrochloride, hydrobromide, hydrogen sulfate, phosphate, maleate, nitrate, besylate and fumarate.

3. The method according to claim 2, wherein said acid addition salt is the fumarate salt.

4. A method for desensitizing a patient to an allergen comprising: administering to the patient one or more allergens and an effective amount of one or more mast cell stabilizers in free or pharmaceutically acceptable acid addition salt form to inhibit or prevent symptoms of mediator release.

5. The method of claim 4, wherein the mast cell stabilizer is selected from the group consisting of cromolyn, cromoglycate, nedocromil, bepotastine, ebastine, epinastine, azelastine, lodoxamide tromethamine, lodoxamide trometamol, pemirolast, olopatadine, ketotifen, norketotifen, 10-OH-norketotifen, 9-OH-norketotifen, 9,10-di-OH-norketotifen and those having the general formula: 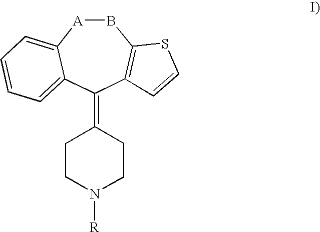
wherein:
a) -A-B— is a moiety having the formula:
i) —CO—CH₂—
ii) —CH₂—CO—
iii) —CH₂—CH₂—
iv) —CHOH—CH₂—
v) —CHOH—CHOH—
vi) —CH₂—CHOH—; or
vii) —CO—CO—; and
b) R is a hydroxyalkyl or a carboxyalkyloxyalkyl moiety; 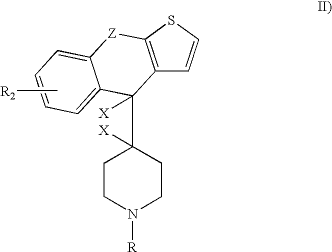
wherein:
a) —Z— is —CH₂—CH₂— or —CH═CH—;
b) R₁ is a H or C₁₋₄alkyl; and
c) R₂ is H or halogen.
or 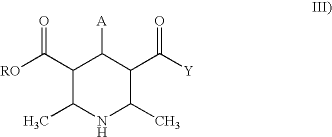
wherein:
a) A is 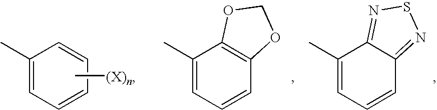
b) n is 1 or 2;
when n is 1, X is H, halogen, CF₃, alkyne, —S(O)_{n′}, OR or NO₂;
when n is 2, X is H or halogen;
c) n′ is 0, 1 or 2;
d) R is C₁₋₅(un)branched alkyl;
e) Y is Z(C(R′)(R′))_{n″}Z′ or 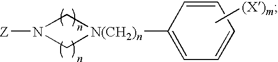
wherein:
i) n″ is 2-4;
ii) m is 0, 1, 2, 3;
iii) R′ is H or R;
iv) X′ is H, halogen, CF₃, alkyne, —S(O)_{n′}R, OR or NO₂;
v) Z is NH or O;
vi) Z′ is N(R′)(R″) or 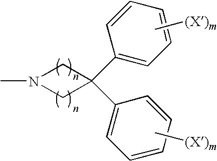
or R″ is 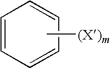
in free or pharmaceutically acceptable acid addition salt form.

6. The method according to claim 5, wherein the one or more allergens is peanut allergen.

7. The method according to claim 4 in combination with a second method for prophylactically treating, controlling, delaying or reducing severe reaction to allergen in a patient at risk of systemic anaphylaxis if exposed to such allergen and for desensitizing a patient to an allergen, wherein the second method comprises: administering to the patient an effective amount of ketotifen in free or pharmaceutically acceptable acid addition salt.

8. A method of allergen desensitization comprising: administering to a patient in need thereof: (i) increasing doses of allergen to induce tolerance, and
(ii) an amount of one or more mast cell stabilizers in free or pharmaceutically acceptable acid addition salt form effective to inhibit symptoms of mediator release;
or an amount of at least one antihistamine to inhibit symptoms of mediator release;
or an amount of a compound having dual antihistamine and mast cell stabilizing activities to inhibit symptoms of mediator release.

9. A method of allergen desensitization comprising administering to a patient in need thereof (i) increasing doses of allergen to induce tolerance and (ii) an amount of at least one antihistamine to inhibit symptoms of mediator release.

10. The method of claim 8, wherein said antihistamine is ketotifen in free or pharmaceutically acceptable salt form.

11. A method of allergen desensitization comprising administering to a patient in need thereof (i) increasing doses of allergen to induce tolerance and (ii) an amount of one or more mast cell stabilizer in free or pharmaceutically acceptable acid addition salt form effective to inhibit symptoms of mediator release.

12. A method of allergen desensitization comprising administering to a patient in need thereof (i) increasing doses of allergen to induce tolerance and (ii) an amount of a compound having dual antihistamine and mast cell stabilizing activities to inhibit symptoms of mediator release.

13. The method of claim 8, wherein said compound is ketotifen in free or pharmaceutically acceptable salt form.

14. The method of claim 8, wherein the method comprises: the step of administering to the patient in need thereof: (i) the increasing doses of allergen to induce tolerance and (ii) the amount of one or more mast cell stabilizers in free or pharmaceutically acceptable acid addition salt form effective to inhibit symptoms of mediator release, wherein said doses of allergen to induce tolerance are higher than would be administered to the patient in the absence of co-administration of a mast cell stabilizer.

15. The method of claim 14, wherein said mast cell stabilizer is ketotifen.

16. The method of claim 1, wherein the allergen is a food allergen.

17. The method of claim 4, wherein the one or more allergens is food, hymenoptera, tree or plant allergen.

18. The method of claim 8, wherein the method comprises: the step of administering to the patient in need thereof:
(i) the increasing doses of allergen to induce tolerance, and
(ii) the amount of the compound having dual antihistamine and mast cell stabilizing activities to inhibit symptoms of mediator release, wherein said doses of allergen to induce tolerance are higher than would be administered to the patient in the absence of co-administration of the compound.

19. The method according to claim 7, wherein the acid addition salt is selected from the group consisting of hydrochloride, hydrobromide, hydrogen sulfate, phosphate, maleate, nitrate, besylate and fumarate.

20. The method according to claim 19, wherein the acid addition salt is the fumarate salt.

21. The method according to claim 7, wherein the mast cell stabilizer is selected from the group consisting of: cromolyn, cromoglycate, nedocromil, bepotastine, ebastine, epinastine, azelastine, lodoxamide tromethamine, lodoxamide trometamol, pemirolast, olopatadine, ketotifen, norketotifen, 10-H-norketotifen, 9-OH-norketotifen, 9,10-di-OH-norketotifen and those having the general formula: 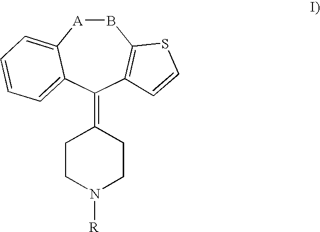
wherein:
a) A-B— is a moiety having the formula:
i) —CO—CH₂—
ii) —CH₂—CO—
iii) —CH₂—CH₂—
iv) —CHOH—CH₂—
v) —CHOH—CHOH—
vi) —CH₂—CHOH—; or
vii) —CO—CO—; and
b) R is a hydroxyalkyl or a carboxyalkyloxyalkyl moiety; 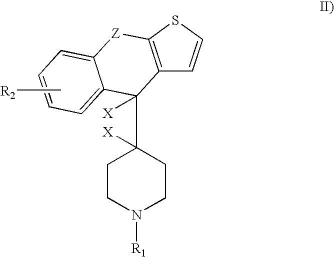
wherein:
a) —Z— is —CH₂—CH₂— or —CH═CH—;
b) R₁ is a H or C₁₋₄alkyl; and
c) R₂ is H or halogen.
or 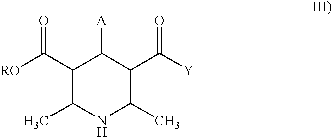
wherein:
a) A is 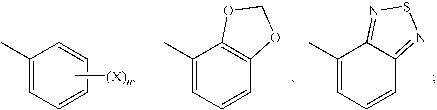
b) n is 1 or 2;
when n is 1, X is H, halogen, CF₃, alkyne, —S(O)_{n′}R, OR or NO₂;
when n is 2, X is H or halogen;
c) n′ is 0, 1 or 2;
d) R is C₁₋₅(un)branched alkyl;
e) Y is Z(C(R′)(R′))_{n″}Z′ or 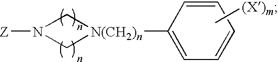
wherein:
i) n″ is 2-4;
ii) m is 0, 1, 2, 3;
iii) R′ is H or R;
iv) X′ is H, halogen, CF₃, alkyne, —S(O)_{n′}, OR or NO₂;
v) Z is NH or O;
vi) Z′ is N(R′)(R″) or 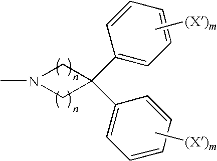
or R″ is 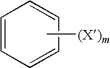
in free or pharmaceutically acceptable acid addition salt form.

22. The method according to claim 21, wherein the allergen is peanut allergen.
